(19) United States
(12) Patent Application Publication
(10) Pub. No.: US 2023/0364573 A1
(43) Pub. Date: Nov. 16, 2023

(54) FIXED BED REACTOR SYSTEM FOR OXIDATIVE DEHYDROGENATION OF ETHANE

(71) Applicant: NOVA CHEMICALS (INTERNATIONAL) S.A., Fribourg (CH)

(72) Inventors: Shahin GOODARZNIA, Calgary (CA); Vasily SIMANZHENKOV, Calgary (CA); Bolaji OLAYIWOLA, Calgary (CA); David GENT, Calgary (CA)

(21) Appl. No.: 18/030,329

(22) PCT Filed: Nov. 5, 2021
(86) PCT No.: PCT/IB2021/060286

§ 371 Date: Apr. 5, 2023

Related U.S. Application Data
(60) Provisional application No. 63/110,385, filed on Nov. 6, 2020.

(51) Int. Cl. B01J 8/04; B01J 8/00; B01J 8/02; C07C 5/333

(52) U.S. Cl. CPC B01J 8/0438; B01J 8/003; B01J 8/0492; B01J 8/0285; C07C 5/3332; B01J 2208/00115; B01J 2208/00752; B01J 2208/00805; B01J 2208/024; B01J 2208/027

(57) ABSTRACT

A fixed bed reactor system for the oxidative dehydrogenation of ethane, comprising a catalyst bed wherein the catalyst capacity profile increases along the length of catalyst bed from the upstream end to the downstream end. The catalyst bed may include one or more sections, across one or more fixed bed reactors, that are identified by a change in catalyst capacity. Catalyst capacity, or the ability to convert ethane into ethylene, may be altered by changing the dilution ratio, void fraction, and or the 35% conversion temperature. A method for loading a fixed bed reactor with an increasing catalyst capacity is also described.

### CLAIM OF PRIORITY

This application claims priority to U.S. Provisional Application No. 63/110,385 filed on Nov. 6, 2020, the entire contents of which are hereby incorporated by reference.

### TECHNICAL FIELD

The present specification is directed to a reactor system for the oxidative dehydrogenation (ODH) of ethane into ethylene.

### BACKGROUND ART

The concept of ODH has been known since at least the late 1960s. Since that time, considerable effort has been expended on improving the process, including improving catalyst efficiency and selectivity. In order for ODH to become a mainstream commercial option, the economic benefit must outweigh the risk associated with potential thermal runway of the reaction. Thermal runway occurs when the exothermic conversion of ethane causes a rapid increase in the catalyst bed temperature that cooling mechanisms are inadequate for responding to lower the temperature. As the catalyst bed temperature spikes the conversion rate of the ethane increases, resulting in a further increase in the catalyst bed temperature, which increases the conversion rate of ethane, and so on. To minimize the risk an operator may choose to limit the catalyst capacity in the reactor and or by increasing the reactor size. These options are not cost effective. The first option reduces the yield of ethylene, and the second option increases capital expenditures incurred for constructing a larger reactor. Provided herein is a reactor system that reduces the maximum catalyst bed temperature without sacrificing conversion and yield.

### SUMMARY OF INVENTION

It has been discovered that reducing the maximum catalyst bed temperature in a fixed bed reactor in a process for oxidative dehydrogenation of ethane can be achieved by loading the catalyst bed to provide an increase in the catalyst capacity along the length of the bed from the upstream end to the downstream end.

In a first aspect there is provided a fixed bed reactor system for the oxidative dehydrogenation (ODH) of ethane to ethylene comprising a catalyst bed, wherein a catalyst capacity profile increases, gradually or in steps, from an upstream end to a downstream end of the catalyst bed.

In additional aspects the catalyst bed comprises at least two non-overlapping catalyst bed sections, arranged in series along the length of the catalyst bed. The catalyst bed sections are identified by a change in the catalyst capacity, each catalyst bed section, except the first catalyst bed section, having a higher catalyst capacity than the immediately preceding catalyst bed section.

The catalyst capacity, or ability to convert ethane in the ethylene, can be assessed by determining the 35% conversion temperature, which is dependent on which and how much catalyst, is present, to what degree the catalyst is diluted within the bed with catalyst additives and or heat dissipative particles, and the void fraction within the catalyst bed. In a second aspect there is provided a process for the oxidative dehydrogenation of ethane comprising introducing a feed stream comprising ethane and oxygen into a fixed bed reactor comprising a catalyst bed having a catalyst capacity that increases, gradually or in steps, from the upstream end to the downstream end, to form a product stream comprising ethylene.

In a third aspect there is provided a method for loading a catalyst bed of a fixed bed reactor for use in a process for the oxidative dehydrogenation comprising:
preparing two or more catalyst bed compositions, the catalyst bed compositions comprising an ODH catalyst;
determining a catalyst capacity for each of the catalyst bed compositions;
separately pouring, in sequential order, the catalyst bed compositions into the fixed bed reactor at a rate slow enough to allow dense and random packing, with the catalyst bed composition having the lowest catalyst capacity poured into the upstream end and the catalyst bed composition having the highest catalyst capacity poured into the downstream end; and securing the poured catalyst bed compositions within the fixed bed reactor to form a loaded catalyst bed; and
   wherein the catalyst bed compositions form distinct catalyst bed sections, the catalyst bed sections identified by the change in catalyst capacity, which increases from the upstream end to the downstream end.

### BRIEF DESCRIPTION OF THE DRAWINGS

To easily identify the discussion of any particular element or act, the most significant digit or digits in a reference number refer to the figure number in which that element is first introduced. The schematic representations of a catalyst bed in accordance with embodiments of the present disclosure are intended to facilitate understanding of the different catalyst bed loading scenarios. The skilled person would appreciate that the size and shape of the catalyst particles and heat dissipative particles in the figures is for demonstration purposes and that the particles are not to scale, and in practice vary in size and shape. For simplicity, labeling is limited to a single instance of the catalyst particles and heat dissipative particles in each figure.

FIG. 1 illustrates a schematic representation of a typical catalyst bed with uniform catalyst capacity.

FIG. 2 illustrates a schematic representation of a catalyst bed with a gradual increase of catalyst capacity in accordance with an embodiment.

FIG. 3 illustrates a schematic representation of a catalyst bed with two sections having different dilution ratios with the same catalyst in accordance with an embodiment.

FIG. 4 illustrates a schematic representation of a catalyst bed with two sections having different catalyst compositions in accordance with an embodiment.

FIG. 5 illustrates a schematic representation of a catalyst bed with two sections having different catalyst compositions in accordance with an embodiment.

FIG. 6 illustrates a schematic representation of a catalyst bed with two sections of different lengths and having different void fractions in accordance with an embodiment.

FIG. 7 illustrates a schematic representation of a catalyst bed with two sections having different catalyst compositions and separated by a region devoid of catalyst in accordance with an embodiment.

FIG. 8 illustrates a schematic representation of a catalyst bed with two contiguous sections separated from a third section by a region devoid of catalyst, the sections differing in void fraction or catalyst composition in accordance with an embodiment.

FIG. 9 illustrates a schematic representation of a catalyst bed with two contiguous sections housed in a first reactor separated from a third section housed in a second reactor, the sections differing in void fraction or catalyst composition in accordance with an embodiment.

FIG. 10 illustrates a temperature profile for examples 1 through 4.

### DESCRIPTION OF EMBODIMENTS

Provided herein is a reactor system for the oxidative dehydrogenation (ODH) of ethane to ethylene. Embodiments of the present reactor system are directed to an increasing catalyst capacity profile along the length of the catalyst bed of the reactor system. Specifically, disclosed herein is a reactor system that comprises a catalyst bed characterized by an increase, from the upstream end to the downstream end of the bed, in the catalyst capacity. The arrangement of a catalyst bed with increasing catalyst capacity along its length provides a mechanism for minimizing the maximum process temperature of the catalyst bed at the upstream end, where ethane and oxygen first contact the ODH catalyst and where the risk of an uncontrollable temperature spike is highest.

Other than in the operating examples or where otherwise indicated, all numbers or expressions referring to quantities of ingredients, reaction conditions, etc. used in the specification and claims are to be understood as modified in all instances by the term “about”. Accordingly, unless indicated to the contrary, the numerical parameters set forth in the following specification and attached claims are approximations that can vary depending upon the properties that the present disclosure desires to obtain. At the very least, and not as an attempt to limit the application of the doctrine of equivalents to the scope of the claims, each numerical parameter should at least be construed in light of the number of reported significant digits and by applying ordinary rounding techniques.

### Definitions

As used herein, the term “catalyst capacity” refers to the ability of the catalyst bed to convert ethane to ethylene and can be used to describe the catalyst bed as a whole, individual catalyst bed sections, or at point along the length of the catalyst bed. The catalyst capacity is dependent on the catalyst composition, the dilution ratio, and the void fraction.

As used herein, the term “catalyst bed” refers to the volume of the region, or regions, occupied by catalyst particles and heat dissipative particles, if present, and including the spaces between such particles. By regions it is intended to mean having a start and end point along the length of the reactor system.

As used herein, the term “catalyst bed length” refers to the length of the catalyst bed beginning from an upstream end, where ethane and oxygen first contact the ODH catalyst, and ending at a downstream end, where the final product stream is formed and past the point where ethane and oxygen may contact active ODH catalyst and excluding regions devoid of catalyst. For example, in a multiple reactor system the catalyst bed length is intended to include the entire length from the upstream end of the first catalyst bed section in the first reactor to the downstream end of the last catalyst bed section in the last reactor in the series but excluding intervening sections devoid of catalyst. Also, for a single reactor system with multiple catalyst bed sections separated by regions devoid of ODH catalyst, the catalyst bed length is intended to cover from the upstream end of the first catalyst bed section to the downstream end of the final catalyst bed section but excluding intervening sections devoid of ODH catalyst.

As used herein, the term “catalyst bed sections” refers to sections or regions within the catalyst bed that can be identified by a change in the catalyst capacity in relation to adjacent catalyst bed sections. Adjacent catalyst bed sections may be contiguous in that they share a common boundary or may be separated by regions devoid of catalyst. Adjacent catalyst bed sections that share a common boundary are generally non-overlapping although some infiltration of catalyst particles from each section may fall beyond the boundary into an adjacent section during loading and settling of the catalyst bed. Catalyst bed sections comprise a uniform distribution of a catalyst bed composition that includes catalyst particles and heat dissipative particles, if present, of similar size and composition, and therefore comprise a uniform dilution ratio and a uniform void fraction.

As used herein, the term “catalyst particles” refers to the particles which are loaded into the catalyst bed and contain active ODH catalyst and includes, if present, any catalyst additives, including, but not limited to, binders, supports, and carriers. Preparing catalyst particles for use in an ODH process falls within the expertise of the person skilled in the art. As used herein, the term “conversion” refers to the percentage of ethane carbon atoms in the feed that are converted to carbonaceous products, and can be calculated according to the formula:

$$Conversion ( \% ) = \frac{\frac{\begin{matrix}Net mass flow rate of converted {C}_{2} {H}_{6} \\ ( g {C}_{2} {H}_{6} / min )\end{matrix}}{\begin{matrix}Molecular weight of {C}_{2} {H}_{6} \\ ( g {C}_{2} {H}_{6} / mol {C}_{2} {H}_{6} )\end{matrix}}}{\frac{\begin{matrix}Mass flow rate of feed {C}_{2} {H}_{6} \\ ( g {C}_{2} {H}_{6} / min )\end{matrix}}{\begin{matrix}Molecular weight of {C}_{2} {H}_{6} \\ ( g {C}_{2} {H}_{6} / mol {C}_{2} {H}_{6} )\end{matrix}}} \times 100$$

where the net mass flow of converted C₂H₆ refers and is equal to the mass flow rate of C₂H₆ in the product stream minus the mass flow rate of C₂H₆ in the feed stream.

As used herein, the term “dilution ratio” refers to degree to which the catalyst is diluted with heat dissipative particles and catalyst additives, such as binders, carriers, and supports, in the catalyst bed as a whole or in an individual catalyst bed section. The dilution ratio is calculated according to the formula:

$$Dilution ratio = \frac{Mass of catalyst bed - Mass of catalyst}{Mass of catalyst bed}$$

As used herein, the term “flammability envelope” refers to the envelope defining the flammability zone in mixtures of fuel (e.g. ethane), oxygen and a heat removal diluent gas.

As used herein, the term “gas hourly space velocity” refers to the ratio of the gas volumetric flow rate where the gas includes the reacting gas species and an optional heat removal diluent gas at standard conditions (i.e., 0° C., 1 bar) to the volume of active phase in the catalyst bed.

As used herein, the term “heat dissipative particles” refers to inert non-catalytic particles can be used within the catalyst bed or one or more of the catalyst bed sections to improve cooling homogeneity and reduction of hot spots by enhancing the rate of radial heat transfer from the catalyst bed or catalyst bed section directly to the walls of the reactor. Heat dissipative particles have the same or higher thermal conductivity compared to the catalyst particles.

As used herein, the term “heat removal diluent gas” refers to a gas that dilutes a stream and can remove heat from the stream.

As used herein, the term “inert metal rods” refers to not catalytically active metal rods, roughly cylindrical in shape, having at least one dimension smaller than the reactor inner diameter. The inert metal rods can include a heat pipe. The inert metal rods can have fins.

As used herein, the “ODH catalyst” refers to a catalyst that catalyzes the conversion, in the presence of oxygen, of ethane into ethylene. The term is intended to cover the final product of catalyst synthesis, prior to and excluding catalyst additives, including, but not limited to, binders, carriers, and supports. Catalysts includes all ODH catalysts known in the art, particularly mixed metal oxide catalysts as described herein. References to “catalyst” or “catalysts” is intended, unless otherwise indicated, to mean ODH catalyst or ODH catalysts, respectively. Reference to an ODH catalyst may also include a mixture of different ODH catalysts (or catalyst species), all capable of converting, in the presence of oxygen, ethane in ethylene. The term “catalyst species” may be used when referring to a catalyst having a specific empirical formula.

As used herein, the term “catalyst capacity profile” refers to the change, along the length of the catalyst bed, of the catalyst capacity.

As used herein, the term “void fraction” refers to the volume of void space or inert space inside the catalyst bed which is not occupied by catalyst particles or heat dissipative particles, divided by the total volume of the catalyst bed. The catalyst bed as a whole or individual sections of the entire catalyst bed may be described as having a void fraction.

As used herein, the term “residence time” refers to a measure of how much time material that is flowing through a volume spends in the volume.

As used herein, the term “weight hourly space velocity” refers to the ratio of the gas mass flow rate where the gas includes the reacting gas species and an optional heat removal diluent gas to the mass of the active phase of the catalyst bed.

### The Fixed Bed Reactor System

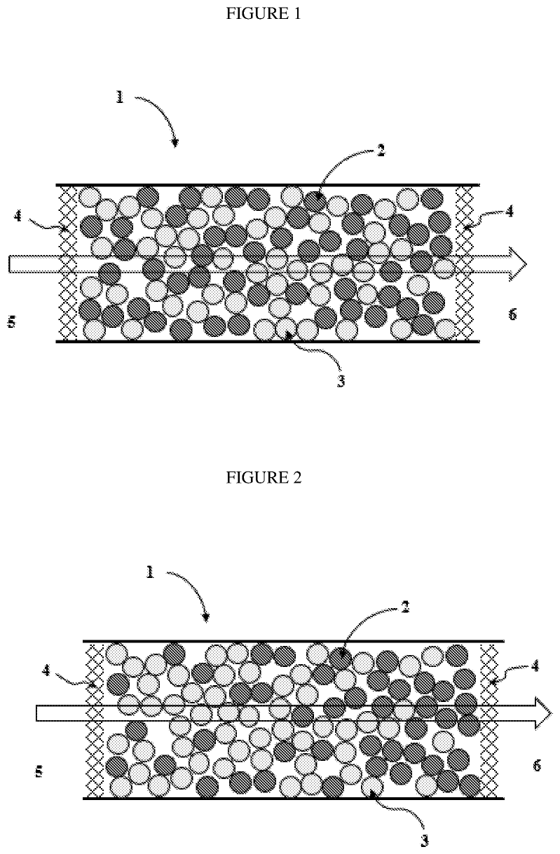

ODH of ethane includes contacting a mixture of ethane and oxygen in one or more ODH reactors with one or more mixed metal oxide catalysts under conditions that promote oxidative conversion of ethane into ethylene and may be performed with a variety of reactor types, including conventional fixed bed reactors, shell-and-tube reactors, and tube reactors. FIG. 1 shows a catalyst bed 1 of an ODH reactor with a uniform distribution of a similar number of similarly sized catalyst particles 2 (dark grey circles) and heat dissipative particles 3 (light grey circles), flanked on each side by a region 4 (hatched) devoid of catalyst particles. The catalyst particles 2 and heat dissipative particles 3 are immobilized and contained by the sides of the reactor or tube and the regions 4. In an ODH of ethane process, ethane and oxygen (indicated by the hollow arrow) are introduced at the upstream end 5 of the reactor or tube and passed through the catalyst bed 1 where conversion occurs, and a product or outlet stream is removed at the downstream end 6 of the reactor or tube. In a tube reactor the catalyst bed is contained within a single tube (the reactor), while in a shell-and-tube reactor the catalyst bed is contained across multiple tubes which are encased in a shell, with coolant flowing between the tubes.

Designing a fixed bed reactor suitable for use with the reactor system disclosed herein can follow techniques known for reactors of this type. A person skilled in the art would know which features are required with respect to shape and dimensions, inputs for reactants, outputs for products, temperature and pressure control, and means for immobilizing the catalyst. While known for use in the ODH of ethane, fluidized beds are not relevant for the present disclosure.

### Maximum Process Temperature

The ODH of ethane generates heat, with the upstream end, where ethane and oxygen first contact the catalyst, typically showing the largest spike in temperature within the catalyst bed. Movement down the length of the bed is accompanied by a decrease in catalyst bed temperature, which levels off to a consistent temperature (isotherm). The maximum process temperature typically occurs within the first 10 to 40% of the length of the catalyst bed, depending on flow rate and or flow velocity. A temperature gradient or temperature differential may exist from the center of the catalyst bed to the walls of the reactor. The temperature of the walls of the reactor resembles the temperature of the coolant which surrounds the reactor, and the ability of the coolant to remove heat from the reactor is tested when there is a larger temperature gradient. The larger the temperature gradient the greater the risk for a thermal runaway. An objective of the present disclosure is to minimize the risk of thermal runaway by minimizing the temperature differential.

ODH reactor systems are designed with cooling mechanisms to extract heat and permit maintenance of a steady-state, or near steady-state, catalyst bed temperature during operations. However, there is still a risk that a spike in temperature may overwhelm cooling capacity, leading to thermal runaway. To avoid this risk an operator may choose to reduce the amount of one or both of ethane and oxygen in the feed, taking into account the amount of catalyst that is loaded into the reactor. The effect is to lower the catalyst capacity as less ethane is converted, and consequently less heat is generated. Unfortunately, this also reduces yield of ethylene which reduces cost effectiveness. A larger reactor using a similar starting amount of ethane to achieve the same yield would theoretically have a lower risk as the feed would be more dilute, owing to a larger volume, but the capital expenditure and downtime may be excessive. Alternatively, a reactor could be reloaded using less of the same catalyst or using a less active catalyst, but the effect is still the same. Lower activity, lower yield.

In the setup described above in relation to FIG. 1 and even though the ethane conversion decreases from the upstream end to the downstream end, the catalyst capacity, or ability to convert ethane into ethylene, along the length of the reactor would be relatively constant, owing to the uniform distribution of catalyst particles of a similar size and heat dissipative particles of similar size, the catalyst particles all containing a similar amount of the same catalyst. The catalyst capacity profile with a uniform distribution of catalyst particles and heat dissipative particles would be constant, or relatively constant, along the length of the catalyst bed.

Embodiments of the present disclosure are directed to a fixed bed reactor system for the oxidative dehydrogenation of ethane into ethylene comprising a catalyst bed comprising an ODH catalyst, wherein the catalyst capacity increases along the length of the catalyst bed. In some embodiments, the catalyst capacity increases gradually from the upstream end to the downstream end of the catalyst bed. In another embodiment, the catalyst capacity increases in one or more steps. The change in catalyst capacity along the length of the catalyst bed, or catalyst capacity profile, is a function of the loading design, or how the catalyst particles are loaded and packed into the bed. The reactor system can comprise a single reactor or multiple reactors.

### Catalyst

To alter the catalyst capacity of a catalyst bed a user may change the amount of catalyst present, or by changing the composition of the catalyst. Changing the amount is straightforward. Changing the composition requires changing the catalyst species that make up the catalyst. For a catalyst that comprises a single species it may involve a simple substitution with a different catalyst species having a different activity, or it may involve adding one or more additional catalyst species to form a catalyst with multiple species. For a catalyst that comprises a mixture of two or more catalyst species it may involve adjusting the contribution of each of the species present in the mixture, removal of a particular species from the mixture, or the addition of a previously absent catalyst species.

Catalyst species with different empirical formulas may have different conversion rates under identical conditions. Comparing different catalysts for their ability to convert ethane into ethylene can be accomplished by determining the temperature at which there is 35% conversion of ethane. Determination of the 35% conversion can be performed by loading the catalyst to be tested into a reactor, passing a feed comprising ethane and oxygen over the catalyst under typical ODH operating conditions to form a product stream, and identifying the temperature at which 35% of the ethane is converted into a product. Loading a large, commercially sized reactor, particularly a shell-and-tube reactor with 1000s of tubes, is time consuming and for the purpose of ascertaining the 35% conversion temperature of the catalyst is not economically feasible. A small-scale reactor, or microreactor unit (MRU), is ideal for determination of and comparison between different catalyst species of the 35% conversion temperature. Comparison between catalyst species requires loading a similar amount, size, and shape of the catalyst species in a similar volume, and testing using identical ODH operating conditions (e.g. feed compositions, pressure, flow rate). A detailed MRU setup is described below, which can be used to assess 35% conversion temperature for individual catalyst species, mixtures of one or more catalyst species, catalyst particles, or representative samples of a catalyst bed or catalyst bed section (catalyst bed compositions).

Catalysts with a lower 35% conversion temperature have a higher ability to convert ethane into ethylene compared to a catalyst with a higher 35% conversion temperature. When comparing two different catalyst beds, each having a uniform distribution of a different catalyst, but with identical, or nearly identical, dilution fractions and void ratios, the catalyst bed having the catalyst with the lowest 35% conversion temperature will have a higher catalyst capacity.

### Dilution Ratio

A catalyst may be diluted in the catalyst bed by combining the catalyst with catalyst additives, such as a support and or binders, to form catalyst particles. Also, catalyst beds may be packed with not only catalyst, or catalyst particles, but also with heat dissipative particles. The dilution ratio, the degree to which the catalyst is diluted with one or both of catalyst additives and heat dissipative particles, impacts the catalyst capacity. The dilution ratio is calculated by dividing the total mass of heat dissipative particles and catalyst additives (e.g. support, binders) by the total mass of the catalyst bed (mass of the catalyst and total mass of heat dissipative particles and catalyst additives). Changing the dilution ratio of a catalyst bed may involve changing one or both of the amount of catalyst relative to heat dissipative particles present in the bed and changing the amount of catalyst additives relative to catalyst in formation of the catalyst particles.

Dilution ratios applicable for use in the fixed bed reactor system disclosed herein may theoretically range from 0.0 to about 0.95. However, with some exceptions, most catalyst species will require a binder in order to maintain structural properties. The minimum amount of binder is generally around 5 wt. % of a complete catalyst including the binder, which, in the absence of heat dissipative particles in the bed, works out to a dilution ratio of 0.05.

Examples of heat dissipative particles include, for example, DENSTONE® 99 (Saint-Gobain Ceramics & Plastics, Inc.) alumina particles, or SS 316 particles, or inert metal rods that can be inserted to create inert space in the catalyst bed. The use of inert non-catalytic heat dissipative particles can be used within one or more of the ODH reactors. The heat dissipative particles can be present within the catalyst bed and include one or more non catalytic inert particulates having a melting point at least about 50° C. above the temperature upper control limit for the reaction, in some embodiments at least about 250° C. above the temperature upper control limit for the reaction, in further embodiments at least about 500° C. above the temperature upper control limit for the reaction. The heat dissipative particles can have a particle size in the range of about 0.5 to about 15 mm, in some embodiments in the range of about 0.5 to about 7.5 mm, in some embodiments in the range of about 1.0 to about 5.0 mm. The heat dissipative particles can have a thermal conductivity of greater than about 30 W/mK (watts/meter Kelvin) within the reaction temperature control limits. In some embodiments the heat dissipative particles are metal alloys and compounds having a thermal conductivity of greater than about 50 W/mK (watts/meter Kelvin) within the reaction temperature control limits. Non-limiting examples of suitable metals that can be used in these embodiments include, but are not limited to, silver, copper, gold, aluminum, steel, stainless steel, molybdenum, and tungsten. The heat dissipative particles can be added to the bed in an amount from about 5 to about 95 wt. %, in some embodiments from about 30 to about 70 wt. %, in other embodiments from about 45 to about 60 wt. % based on the entire weight of the bed. The particles are employed to potentially improve cooling homogeneity and reduction of hot spots in the bed by transferring heat directly to the walls of the reactor. The heat dissipative particles can optionally be pressed or extruded with the catalyst in formation of catalyst particles.

Lowering the dilution ratio in a catalyst bed has the effect of increasing catalyst capacity. When comparing two different catalyst beds, each having a uniform distribution of the same catalyst and identical, or nearly identical void fractions, the catalyst bed with the lower dilution ratio will have a higher catalyst capacity.

### Void Fraction

Packing a catalyst bed with catalyst particles and possibly heat dissipative particles creates space between the particles, or void fraction. The void fraction can be altered by changing the size and shape of the catalyst particles and or the heat dissipative particles. For example, larger particles create more void space. Also, ring shaped catalyst particles have a higher void fraction than discs of similar diameter and thickness. Determining the void fraction falls within the purview of the person skilled in the art. The method of choice for measuring void fraction is not critical, provided that the same method is used when comparing catalyst bed compositions.

The void fraction can be determined by calculation, using the sizes, shapes, and amounts of each of the components in the catalyst bed. Software programs are available for calculating the void fraction when the sizes and shapes of the particles are known, and assuming a random packing. The void fraction may also be measured by dispensing a sample of the catalyst bed into a container at room temperature and atmospheric pressure, to the full capacity of the container, and then filling the container with a low viscosity fluid. The void fraction can then be determined by dividing the amount of low viscosity fluid required to fill the container by the volume of the container. Choosing a low viscosity fluid that does not enter catalyst bed components to a significant degree (absorbed into pores), or dissolves catalyst bed components, falls within the expertise of the person skilled in the art. Suitable examples include, but are not limited to, oil (for hydroscopic catalyst bed components) and water (for hydrophobic catalyst bed components). The low viscosity fluid should be given time to diffuse throughout the material and to allow air bubbles to leave the bed, typically around 15 minutes. It is important that the catalyst bed fills the container to capacity to mimic the loading and packing within a reactor. Any size of container may be used, provided the size does allows for packing similar to that of the reactor. For example, if the catalyst bed is to be packed into a reactor tube with an internal diameter of 0.5″ then a cylindrical container with a 0.5″ internal diameter may be ideal. The length of the cylinder ideally is 6″ or greater.

In some embodiments of the present disclosure the void fraction is from 0.30 to 0.70. In some embodiments of the present disclosure the void fraction is from 0.30 to 0.60. In some embodiments of the present disclosure the void fraction is from 0.40 to 0.50.

By altering the void fraction an operator is reducing the surface area of the catalyst per fixed volume of the catalyst bed. The effect is to reduce the number of active sites on the surface of the catalyst, where the majority of conversion of ethane into ethylene occurs, which reduces catalyst capacity. When comparing two different catalyst beds, each having a uniform distribution of similar catalyst particles and similar dilution fraction, but having different void fractions, the catalyst bed with the lower void fraction will have a higher catalyst capacity.

### Gradual Increase

FIG. 2 illustrates a catalyst bed 1 where the dilution ratio gradually decreases along the length of the catalyst bed. As shown in FIG. 2 the frequency of heat dilutive particles 3 gradually decrease while catalyst particles 2 increase in frequency along the bed from the upstream end 5 to the downstream end 6. The effect is to increase the catalyst capacity from the upstream end to the downstream end.

In some embodiments of the disclosure the catalyst capacity increases gradually along the length of the catalyst bed due to a gradual decrease in the dilution ratio.

It is also contemplated to provide a catalyst bed where the 35% conversion temperature gradually increases from the upstream end to the downstream end. The 35% conversion temperature can be increased gradually by using a mixture of catalyst particles, each with a different 35% conversion temperature. The catalyst particles with the higher 35% conversion temperature may decrease in frequency from the upstream end to the downstream end, while the catalyst particles with the lower 35% conversion temperature may increase. The 35% conversion temperature for a catalyst bed section is the average of the 35% conversion temperatures of the different catalyst particle types, accounting for weight fraction, at each point along the length of the catalyst bed section.

In some embodiments of the disclosure the catalyst capacity increases gradually along the length of the catalyst bed due to a gradual decrease in the 35% conversion temperature of the catalyst particles.

It is also contemplated to provide a catalyst bed where the void fraction gradually decreases from the upstream end to the downstream end. The void fraction can be decreased gradually by using a mixture of catalyst particles, each with a different size and or shape. The catalyst particles that pack less tightly, creating more void space, may decrease in frequency from the upstream end to the downstream end, while the catalyst particles that pack more tightly may increase.

In some embodiments of the disclosure the catalyst capacity increases gradually along the length of the catalyst bed due to a gradual decrease in the void fraction.

### Catalyst Capacity Steps

Shell-and-tube reactors may contain thousands of tubes, where loading a catalyst bed with a gradual increase in catalyst capacity along the length of each tube, while feasible and likely beneficial, would be logistically difficult and costly. Development for a method for loading thousands of tubes with an increasing catalyst capacity profile in a cost-effective manner would be beneficial. Practically speaking, however, it is likely simpler and more cost efficient to separate the catalyst bed into sections with different uniform catalyst capacities, with a first upstream section followed by one or more subsequent sections, each section having an upstream end and a downstream end, ending with a final downstream section. With the exception of the first upstream section and the final downstream section, each section can be an upstream section or a downstream section in relation to adjacent sections. For example, in a four-section catalyst bed, the second section is the downstream section to the first upstream section and the upstream section to the third section, and the third section is the downstream section to the second section and the upstream section to the final downstream section. The sections may be contiguous or be separated by regions devoid of catalyst.

In some embodiments, the catalyst bed comprises at least two non-overlapping catalyst bed sections arranged in series along the catalyst bed length, each catalyst bed section having an upstream end and a downstream end, wherein a first upstream catalyst bed section is followed by one or more downstream sections with the last catalyst bed section ending at the downstream end of the catalyst bed, and wherein the catalyst bed sections are identified by a change in the catalyst capacity with each catalyst bed section having a higher catalyst capacity than the preceding upstream catalyst bed section.

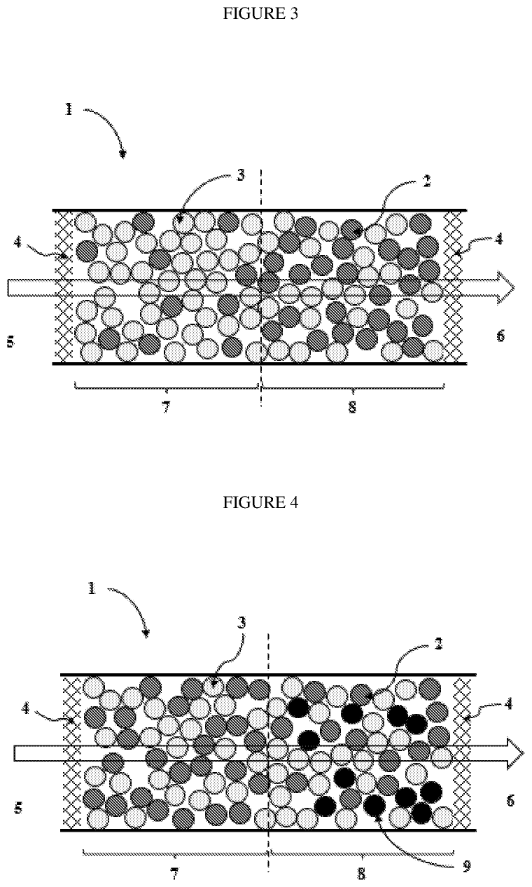

FIG. 3 is a schematic representation of a catalyst bed 1 that is housed within a single reactor and includes an upstream section 7 and a downstream section 8 (indicated by brackets). The sections are contiguous with the change in catalyst capacity indicated by a dashed line. Each section spans approximately half the length of the catalyst bed and are packed with a similar total number of catalyst particles and heat dissipative particles of a similar size. The dilution fraction in upstream section 7 is higher than in downstream section 8, owing to the presence of a larger number of heat dissipative particles 3 as compared to catalyst particles 2 in that section. The catalyst capacity in upstream section 7 is lower than in downstream section 8.

In some embodiments of the present disclosure, one or more catalyst bed sections comprise a dilution ratio that is lower than the preceding catalyst bed section.

In some embodiments of the present disclosure, one or more catalyst bed sections comprise a dilution ratio that is lower than the preceding catalyst bed section, wherein the catalyst bed sections comprise a similar amount of the same catalyst.

The dilution ratio for a catalyst bed section may range from 0, where there are no heat dissipative particles or catalyst additives, to 0.95, where heat dissipative particles and catalyst additives comprise 95% of the mass of the catalyst bed section. Packing a bed with nothing but catalyst, while possible, may be limiting technically Preferably, the dilution ratio of the catalyst bed sections ranges from 0.30 to 0.9, more preferably 0.50 to 0.80.

In some embodiments of the present disclosure, the reactor system comprises one or more catalyst bed sections having a dilution ratio of from 0.00 to 0.95.

In some embodiments of the present disclosure, the reactor system comprises one or more catalyst bed sections having a dilution ratio of from 0.30 to 0.90.

In some embodiments of the present disclosure, the reactor system comprises one or more catalyst bed sections having a dilution ratio of from 0.50 to 0.80.

It is expected that with larger differences in the dilution ratio between catalyst bed sections there will be a corresponding larger effect on the maximum process temperature, and consequently, the temperature differential. The largest effect would occur when an upstream catalyst bed section having a dilution ratio of 0.75 is followed by a downstream catalyst bed section with a dilution ratio of 0 (no heat dissipative particles), which is 100% lower. It is contemplated that even small differences may prove to be beneficial, including differences as low as 2%.

In some embodiments, one or more catalyst bed sections comprise a dilution ratio that is from 2 to 100% lower than the preceding section.

In some embodiments, one or more catalyst bed sections comprise a dilution ratio that is from 5 to 70% lower than the preceding section.

In some embodiments, one or more catalyst bed sections comprise a dilution ratio that is from 10 to 50% lower than the preceding section.

### 35% Conversion Temperature

FIG. 4 is a schematic representation of a catalyst bed 1 that is housed within a single reactor and includes an upstream section 7 and a downstream section 8. The sections are contiguous with the change in catalyst capacity indicated by a dashed line. The sections are of a similar size, each packed with a similar total number of catalyst particles and heat dissipative particles and covering approximately half of the length of the bed. The 35% conversion temperature in upstream section 7 is higher than in downstream section 8, owing to the presence of stronger catalyst particles 9 (black circles) having a lower 35% conversion temperature than catalyst particles 2. The 35% conversion temperature in downstream section 8, which comprises a mixture of catalyst particles 2 and stronger catalyst particles 9, on average is lower than the 35% conversion temperature in upstream section 7, in which the catalyst particles are entirely catalyst particles 2. With this loading design downstream section 8 comprises a higher catalyst capacity.

In some embodiments of the present disclosure, one or more catalyst bed sections comprise a 35% conversion temperature that is lower than the preceding catalyst bed section.

In some embodiments of the present disclosure, one or more catalyst bed sections comprise a catalyst having a 35% conversion temperature that is lower than the catalyst in the preceding catalyst bed section, wherein the catalyst bed sections have similar dilution ratios and void fractions.

In some embodiments the reactor system consists of an upstream bed section and a downstream bed section, the upstream bed section and the downstream bed section having similar dilution ratios and void fractions, and wherein the catalyst in the downstream bed section has a higher 35% conversion temperature than the catalyst in the upstream bed section.

In some embodiments the reactor system consists of an upstream bed section and a downstream bed section, the upstream bed section and the downstream bed section having similar dilution ratios and void fractions, and wherein the catalyst in the downstream bed section has a higher 35% conversion temperature than the catalyst in the upstream bed section, and wherein the catalyst in one or both of the upstream bed section and the downstream bed section comprises two or more catalyst species.

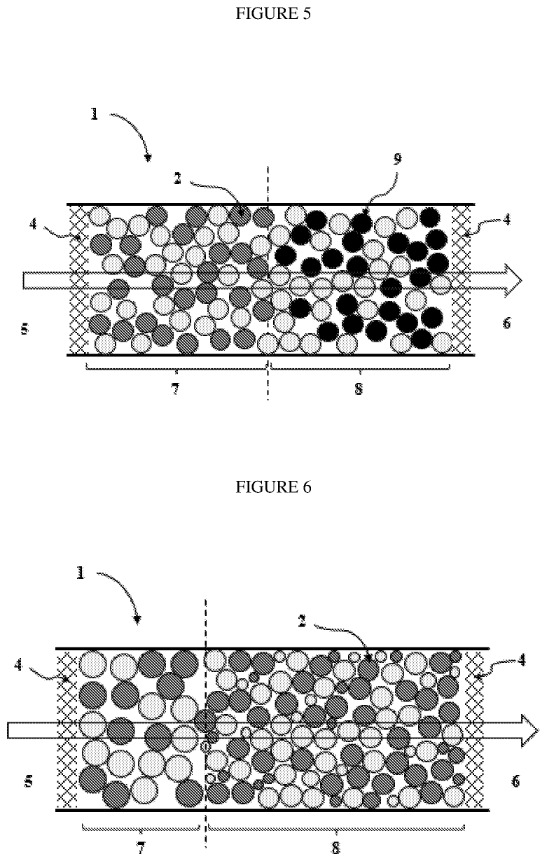

FIG. 5 is a schematic representation of a catalyst bed 1 that is housed within a single reactor and includes an upstream section 7 and a downstream section 8. The sections are contiguous with the change in catalyst capacity indicated by a dashed line. The sections are of a similar size, each packed with a similar number of catalyst particles and heat dissipative particles per volume and covering approximately half of the length of the bed. The 35% conversion temperature in upstream section 7 is higher than in downstream section 8, owing to the presence of stronger catalyst particles 9 (black circles) having a lower 35% conversion temperature than catalyst particles 2. With this loading design downstream section 8 comprises a higher catalyst capacity. Upstream section 7 and downstream 8 may be of different sizes, such that the fraction of the length of the bed is unevenly split between the two sections.

In some embodiments the reactor system consists of an upstream bed section and a downstream bed section, the upstream bed section and the downstream bed section having similar dilution ratios and void fractions, and wherein:
the catalyst in the downstream bed section has a higher 35% conversion temperature than the catalyst in the upstream bed section;
wherein the catalyst in one or both of the upstream bed section and the downstream bed section comprises two or more catalyst species; and
the upstream bed section and downstream bed section comprise from 0.2 to 0.8 of the length of the catalyst bed.

FIG. 6 is a schematic representation of a catalyst bed 1 that is housed within a single reactor and includes an upstream section 7 and a downstream section 8. The sections are contiguous with the change in catalyst capacity indicated by a dashed line. The sections are unequal in size, with the upstream section 7 spanning approximately the first third and the downstream section 8 spanning the final two thirds of the length of the catalyst bed. Upstream section 77 comprises catalyst particles and heat dissipative particles of a larger size than the catalyst particles and heat dissipative particles in downstream section 8. Furthermore, the catalyst particles and heat dissipative particles of downstream section 8 are not only smaller but comprise a variety of sizes. As a result, downstream section 8 is more tightly packed and comprises a much smaller void fraction. The amount of catalyst per volume and the dilution ratios of the sections are similar, so the catalyst capacity of downstream section 8 is higher than upstream section 7.

In some embodiments of the present disclosure, one or more catalyst bed sections comprise a void fraction that is lower than the preceding catalyst bed section.

In some embodiments the reactor system consists of an upstream bed section and a downstream bed section, the upstream bed section and the downstream bed section having a similar type and amount of catalyst and similar dilution ratios and void fractions, and wherein the void fraction of the upstream section is higher than the downstream section.

Similar to the dilution ratio it is expected that a larger difference in the void fraction between catalyst bed sections will have a more significant effect on the maximum process temperature and temperature differential. The largest effect would occur when an upstream catalyst bed section having the largest void fraction of 0.7 is followed by a downstream catalyst bed section with the lowest possible void fraction of 0.3, which is 57.1% lower. It is contemplated that even small differences may prove to be beneficial, including differences as low as 2.0%. In some embodiments, one or more catalyst bed sections comprise a void fraction that is from 2.0 to 57% lower than the preceding section.

In some embodiments of the present disclosure, one or more catalyst bed sections comprise a void fraction that is from 5.0 to 45% lower than the preceding catalyst bed section.

In some embodiments of the present disclosure, one or more catalyst bed sections comprise a void fraction that is from 10 to 25% lower than the preceding catalyst bed section.

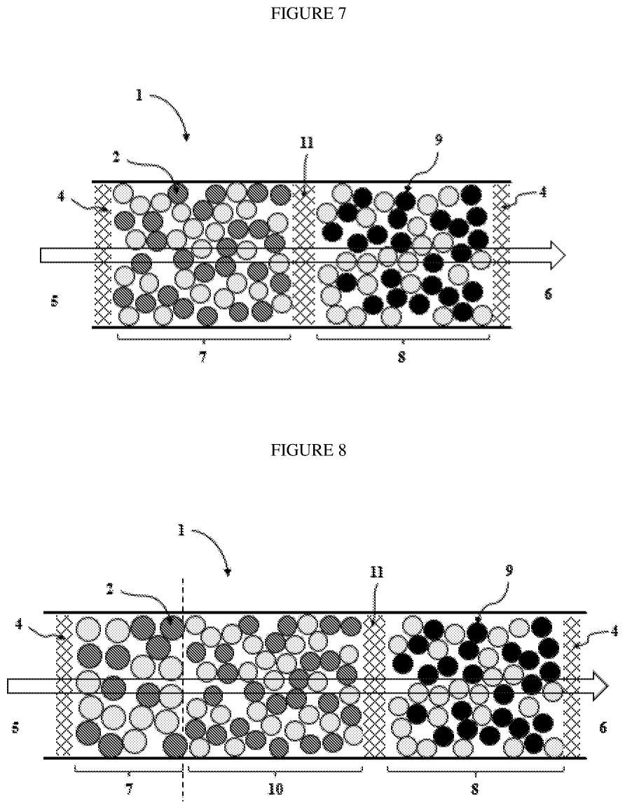

Catalyst bed sections may be separated by regions devoid of catalyst. FIG. 7 and FIG. 8. are schematic representations of a catalyst bed that is housed within a single reactor. In FIG. 7 there is an upstream section 7 and a downstream section 8, and in FIG. 8. there is an additional middle section 10 that is flanked by upstream section 7 and downstream section 8. In FIG. 7 an intervening region 11 devoid of catalyst separates the two sections, while in FIG. 8, the intervening region 11 separates upstream section 7 from middle section 10. The intervening regions 11 are similar to regions 4 in that they can provide support for the catalyst bed section by immobilizing the components, preventing shifting during operation. The intervening regions can be any material that permits passage of feed and product gases through the reactor, passing from one bed section to the next. Examples of intervening regions include, but are not limited to, sections of heat dissipative particles, partitioning plates, static mixers, or any material or structure that prevents catalyst particles and heat dissipative particles from passing between sections. For example, in a vertically oriented reactor, a partitioning plate with holes having diameters that are too small for catalyst particles to pass, preventing the catalyst particles from falling into the lower section while permitting the process gases to pass. Choosing a suitable material or design of an intervening region falls within the expertise of the person skilled in the art.

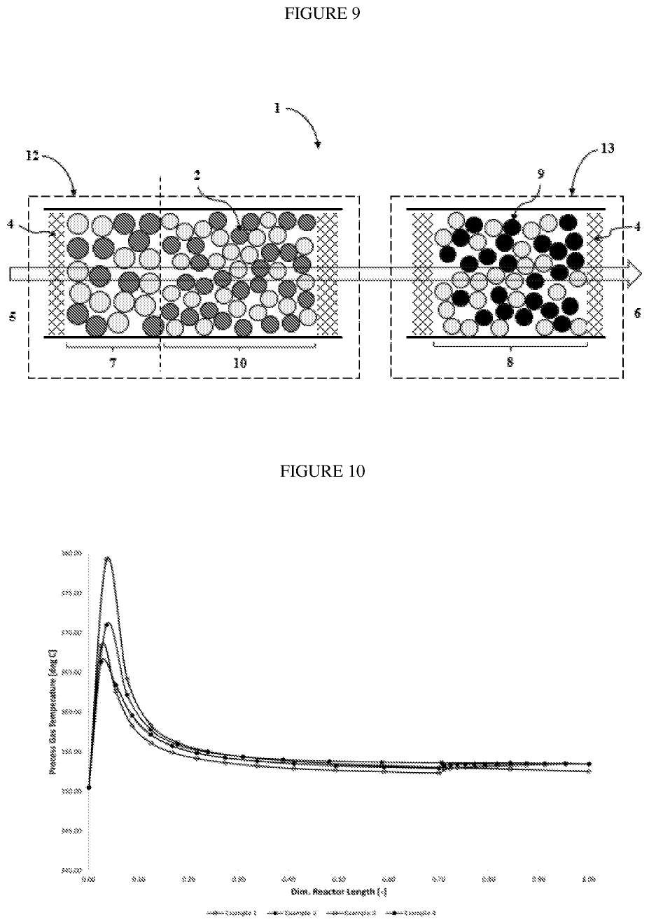

The catalyst bed may include catalyst bed sections that are spread across one or more reactors, each reactor comprising one or more catalyst bed sections. FIG. 9 is a schematic representation of a catalyst bed spread across two reactors (indicated by dotted boxes), with the first reactor 12 comprising two catalyst bed sections, upstream section 7 and middle section 10, and the second reactor 13 comprising downstream section 8. In this scenario the middle section 10 is separated from downstream section 8 by the connection between the first and second reactor. The sections in FIG. 9 are in series, with the upstream section 7 having the lowest catalyst capacity due to a larger void fraction than middle section 10, which has an intermediate catalyst capacity. Downstream section 8 has the highest catalyst capacity as it comprises a similar void fraction to middle section 10 and stronger catalyst particles 9.

In some embodiments the reactor system comprises two or more catalyst bed sections spread across two or more reactors.

In some embodiments the reactor system comprises two or more catalyst bed sections, wherein the catalyst bed sections comprise different catalysts.

### Method of Preparing a Fixed Bed Reactor

Loading a reactor with a fixed bed falls within the knowledge of the person skilled in the art. Operators typically choose a particular type, size, and shape of the catalyst particles, including whether the catalyst particles include catalyst additives, and the type, size, and amount of heat dissipative particles. Before loading, the catalyst bed composition is prepared by mixing all the components to promote uniform distribution. Typically, fixed bed reactors for ODH are vertically oriented and the catalyst bed composition is simply poured, by hand or by using robotic means, into the tube, or tubes for a shell-and-tube reactor, at a rate slow enough to allow dense packing. The catalyst bed components—the catalyst particles and heat dissipative particles—are permitted to settle naturally. The result is a fixed bed reactor with a catalyst bed having a uniform distribution of catalyst particles and heat dissipative particles, the catalyst bed having a uniform catalyst capacity. Expertise in loading reactors in this fashion are common.

Provided herein is a method for loading a catalyst bed in a fixed bed reactor where the catalyst bed comprises one or more non-overlapping sections, arranged in sequence in order of increasing catalyst capacity from the upstream end to the downstream end of the catalyst bed.

Provided herein is a method for loading a catalyst bed in a fixed bed reactor for oxidative dehydrogenation of ethane, the fixed bed reactor comprising an upstream end and a downstream end, the method comprising;
preparing two or more catalyst bed compositions, the catalyst bed compositions comprising an ODH catalyst;
determining a catalyst capacity for each of the catalyst bed compositions;
separately pouring, in sequential order, the catalyst bed compositions into the fixed bed reactor at a rate slow enough to allow dense and random packing, with the catalyst bed composition having the lowest catalyst capacity poured into the upstream end and the catalyst bed composition having the highest catalyst capacity poured into the downstream end; and
securing the poured catalyst bed compositions within the fixed bed reactor to form a loaded catalyst bed; and
   wherein the catalyst bed compositions form distinct catalyst bed sections, the catalyst bed sections identified by the change in catalyst capacity and increasing from the upstream end to the downstream end.

Provided herein is a method for loading a catalyst bed in a fixed bed reactor comprising one or more tubes, each tube having an upstream end and a downstream end, the method comprising;
preparing two or more catalyst bed compositions, the catalyst bed compositions comprising an ODH catalyst;
assessing a catalyst capacity for each of the catalyst bed compositions and ordering the catalyst bed compositions from lowest relative catalyst capacity to highest relative catalyst capacity;
separately pouring, in sequential order, the catalyst bed compositions into the one or more tubes of the fixed bed reactor at a rate slow enough to allow dense and random packing, with the catalyst bed composition having the lowest catalyst capacity poured into the upstream end and the catalyst bed composition having the highest catalyst capacity poured into the downstream end; and
securing the poured catalyst bed compositions within the one or more tubes;
   wherein the catalyst bed compositions form distinct catalyst bed sections, the catalyst bed sections identified by the change in catalyst capacity and increasing from the upstream end to the downstream end.

Preparation of a catalyst bed composition falls within the knowledge of the person skilled in the art. For designing a fixed bed reactor with one or more sections having differing catalyst capacities an operator may vary the relevant factors of catalyst composition, dilution ratio, and or void fraction. In order to assess the differences in catalyst capacity of catalyst bed compositions an operator may consider comparing the properties and predicting which composition will have a higher catalyst capacity. This may be straightforward if two of the factors are identical. For example, it may be obvious that catalyst bed compositions having the same catalyst composition and dilution ratio, but vastly different void fractions will differ in catalyst capacity, with the catalyst bed composition having the smallest void fraction having the greater catalyst capacity. Predictions may be simple particularly if the difference is significant in the one relevant factor. However, when two or more of the factors are different predictions may or may not be reliable. It is preferable to compare the relative 35% conversion temperatures of the catalyst bed compositions as a whole.

In some embodiments of the present disclosure, catalyst capacity is assessed by ordering the catalyst bed compositions by relative 35% conversion temperatures, with the highest relative 35% conversion temperature corresponding to the catalyst bed composition with the lowest relative catalyst capacity.

Assessing the 35% conversion temperature of a catalyst bed composition may involve loading a mini-reactor unit with a sample of the catalyst bed composition and passing a feed stream through the reactor while monitoring the temperature within the reactor and the conversion rate of ethane. Different process conditions, such as the feed composition, pressure, and flow rates, may produce different values of 35% conversion temperature for a particular catalyst bed composition. By “relative” catalyst capacity it is meant that the actual 35% conversion temperature, while relevant, is not essential for comparing two or more catalyst bed compositions. It is more important to compare the 35% conversion temperature of each of the compositions relative to the other compositions so that ordering can be established.

An MRU may include a reactor tube made from stainless-steel tubing (e.g. SWAGELOK® Tubing), with a size that allows packing of the catalyst bed composition that would mirror packing in the fixed bed reactor in which the catalyst bed compositions are intended to be loaded for use in an ODH process. Ideally, the MRU reactor tube shares the same internal and external diameters of the tube or tubes of the target fixed bed reactor. The length of the MRU tube, while not essential, should be long enough to permit steady state operations. Lengths ranging from 6 inches to 3 feet may be ideal. A moveable or multipoint thermocouple (for example a 6-point WIKA Instruments Ltd. K-type thermocouple) may be inserted through the MRU reactor tube and used to measure and control the temperature within the catalyst bed. A room temperature stainless steel condenser may be located after the MRU reactor tube to collect water and acetic acid condensates. The gas product flow may be directed to a gas chromatograph (for example, GC; Agilent 6890N Gas Chromatograph, Using Chrom Perfect-Analysis, Version 6.1.10 for data evaluation) to monitor conversion and selectivity by measuring the levels of the different chemical species present in the product stream.

Samples of the catalyst bed compositions are tested separately by loading the compositions, slowly to ensure dense packing, into the MRU reactor tube. A pre-mixed feed gas, comprising ethane and oxygen and possibly an inert diluent, may be fed to the reactor at standardized conditions for flow and pressure. The feed composition and standardized conditions may be chosen by the operator to approximate the conditions for a typical ODH process and must be identical for testing all catalyst bed compositions in order to properly assess the “relative” 35% conversion temperatures. A typical feed composition may include 20 mol. % ethane, 10 mol. % oxygen, and 70 mol. % inert diluent (e.g. nitrogen). Pressure may be ambient and the flow rate may be held steady at a WHSV of from 2.0 to 3.5 h⁻¹. The temperature may be controlled and increased gradually while monitoring the conversion rate of ethane. The 35% conversion temperature is the temperature at 35% conversion during steady state operations.

Loading a fixed bed reactor as described herein allows for a method of controlling or limiting the maximum process temperature under steady state operating conditions. Cooling systems for an ODH process typically are designed relative to the process isotherm, where temperatures close to the isotherm are easily controlled. Shell-and-tube reactors with tubes having a larger diameter, compared to smaller diameter tubes, have potential to increase the yield of ethylene. However, a larger tube increases the temperature difference between the inner core of the catalyst bed (where the temperature is the highest) and the wall of the tube. Coolant temperatures approach the isotherm temperature and approximate the temperature of the wall of tube, Temperature spikes, or regions where the catalyst bed temperature exceeds the isotherm temperature pose a risk for thermal runaway if the difference is greater than the capacity for the cooling system to remove heat. Typically, maximum process temperatures are observed in the first 20% of the catalyst bed length, where exothermic conversion is highest, releasing heat. The temperature difference between the maximum reaction temperature within a first section of an oxidative dehydrogenation reactor catalyst bed and the temperature in subsequent catalyst bed sections can be from about 1 to about 50° C., or can be from about 2 to about 30° C., in some cases from about 5 to about 20° C. Typically speaking, reactor tubes with a larger diameter demonstrate temperature differential. A larger diameter tube provides an opportunity for increasing the yield but is accompanied by the risk of thermal runaway associated with a large temperature differential. Loading a larger diameter reactor tube with an increase catalyst capacity provides an opportunity for greater yields without the risk of thermal runaway.

Reducing the catalyst capacity in the upstream sections reduces conversion and the associated exothermic release of heat, minimizing the risk of an uncontrollable temperature spike. Furthermore, conditions may allow for the downstream sections to contribute more to conversion, as more ethane is available compared to a scenario where the upstream sections have a similar catalyst capacity and deplete the feed ethane to a low level before it reaches the more downstream sections. Finally, another potential benefit is that having higher catalyst capacity at the downstream end may provide an opportunity to consume any residual oxygen, lowering the oxygen levels in the product stream and potentially avoiding risks associated with processing in the presence of oxygen. Product streams typically are passed through a separation train including a carbon dioxide removal stage with an amine tower which is sensitive to oxygen, and oxygen accumulation within the separation train may form an explosive mixture.

It is conceivable but impractical to vary the catalyst capacity profile among the different tubes in a shell-and-tube reactor, as the variation in isotherm between the tubes would impose cooling control difficulties on an operator.

### The ODH Process

The fixed bed reactor system described herein can be utilized for an ODH process, typical conditions for which are described below. Conditions within the reactor are controlled by the operator and include, but are not limited to, parameters such as temperature, pressure, and flow rate. Conditions will vary and can be optimized for a particular ethane/oxygen feed composition, or for a specific mixed metal oxide catalyst, or whether a heat removal diluent gas is used in the mixing of the reactants. ODH reactors that dehydrogenate ethane to ethylene include at least one feed stream containing oxygen and not less than 20 vol. % of ethane, and at least one outlet stream comprising ethylene, unreacted ethane, one or more carboxylic acids, water, and oxygen.

Use of an ODH reactor for performing an ODH process consistent with the present disclosure falls within the knowledge of the person skilled in the art. The ODH of ethane may be conducted such that the maximum process temperature is from about 300° C. to about 450° C., in some cases from about 300° C. to about 425° C., in other cases from about 300° C. to about 400° C., in some instances from about 310° C. to about 350° C., and at pressures from about 0.5 to about 100 psig (3.447 to 689.47 kPag), in some cases from about 15 to about 50 psig (103.4 to 344.73 kPag), and the residence time, in which the volume of active mixed metal oxide catalyst is in the numerator and the flow rate of feed gas is in the denominator, in the ODH reactor can be from about 0.002 to about 30 seconds, in some cases from about 1 to about 10 seconds.

In embodiments, the ODH process has a selectivity for ethylene of greater than about 85%, in some cases greater than about 90%. The flow of reactants and heat removal diluent gas can be described in any number of ways known in the art. Typically, flow is described and measured in relation to the volume of all feed gases (reactants and diluent) that pass over the volume of the active catalyst bed in one hour, or gas hourly space velocity (GHSV). The GHSV can range from about 50 to about 10000 h⁻¹, in some cases the range is about 500 h⁻¹ to about 1000 h⁻¹. The flow rate can also be measured as weight hourly space velocity (WHSV), which describes the flow in terms of the weight, as opposed to volume, of the gases, excluding heat removal diluent, that flow over the weight of the active catalyst per hour. The WHSV may range from about 0.5 h⁻¹ to about 18.75 h⁻¹, in some cases from about 1.0 to about 10.0 h⁻¹.

The flow of gases through the ODH reactor may also be described as the linear velocity of the gas stream (m/s), which is defined in the art as the flow rate of the gas stream divided by the cross-sectional surface area of the reactor all divided by the void fraction of the mixed metal oxide catalyst bed. The flow rate generally means the total of the volumetric flow rates at standard temperature and pressure (i.e., 0° C. and 1 bar) of all the gases entering the reactor, and is measured where the oxygen and ethane first contact the mixed metal oxide catalyst and at the temperature and pressure at that point. The cross-section of the ODH reactor is also measured at the entrance of the mixed metal oxide catalyst bed. The linear velocity can range from about 5 cm/sec to about 1500 cm/sec, in some cases from about 10 cm/sec to about 500 cm/sec.

The space-time yield of ethylene (productivity) in g/hour per kg of the mixed metal oxide catalyst will often be not less than about 200, in some cases not less than about 500, in other cases not less than about 900, in some instances greater than about 1500, in other instances greater than about 3000, in some situations greater than about 3500 at about 350 to about 400° C. It should be noted that the productivity of the mixed metal oxide catalyst will increase with increasing temperature until the selectivity is decreased.

Mixtures of ethane with oxygen in many cases contain ratios that fall outside of the flammability envelope. For example, a ratio of ethane to oxygen may fall outside the upper flammability envelope. In this instance the percentage of oxygen in the mixture is not greater than about 30 vol. %, in some cases not greater than about 25 vol. %, in other cases not greater than about 20 vol. %. This percentage of oxygen in the mixture depends on the temperature to the reactor inlet, since in many cases the conditions are to stay outside of the flammability limits before entering the reactor tubes. In the reactor tubes the oxygen can be within the flammability envelope, but the catalyst bed itself can act as a flame arrestor. If preheating is done all the way to the reaction temperature, the number can be as low as about 10% oxygen.

With higher oxygen percentages it can be the case to choose ethane percentages that keep the mixture outside of the flammability envelope. While a person skilled in the art would be able to determine an appropriate level it is recommended that the percentage of ethane not exceed 40 vol. %. For instances where the mixture of gases prior to ODH contain 20 vol. % oxygen and 40 vol. % ethane, the balance must be made up with a heat removal diluent gas, such as one or more of nitrogen, carbon dioxide, and steam. The heat removal diluent gas should exist in the gaseous state in the conditions within the reactor inlet and the reactor and should not increase the flammability of the hydrocarbon added to the reactor, characteristics that a skilled worker would understand when deciding on which heat removal diluent gas to employ. Heat removal diluent gas can be added to either of the ethane containing gas or the oxygen containing gas prior to entering the ODH reactor or may be added directly into the ODH reactor.

Mixtures that fall within the flammability envelope are not ideal but may be employed in instances where the mixture exists in conditions that prevent propagation of an explosive event. That is, the flammable mixture is created within a medium where ignition is immediately quenched. For example, a user may design a reactor where oxygen and the ethane are mixed at a point where they are surrounded by flame arresting material. Any ignition would be quenched by the surrounding material. Flame arresting material includes but is not limited to metallic or ceramic components, such as stainless-steel walls or ceramic supports. Another possibility is to mix oxygen and ethane at a low temperature, where an ignition event would not lead to an explosion, then introduce into the reactor before increasing the temperature. The flammable conditions do not exist until the mixture is surrounded by the flame arrestor material inside of the reactor.

### ODH Catalyst

Any of the mixed metal oxide catalysts used as ODH catalysts known in the art are suitable for use in the methods disclosed herein. Non-limiting examples of suitable oxidative dehydrogenation catalyst include those containing one or more mixed metal oxides selected from:

i) catalysts of the formula:

MoₐV_{b}Te_{c}Nb_{d}PdₑO_{f}

wherein a, b, c, d, e and f are the relative atomic amounts of the elements Mo, V, Te, Nb, Pd and O, respectively; and when a=1, b=0.01 to 1.0, c=0.01 to 1.0, d=0.01 to 1.0, 0.00<e<0.10 and f is a number to at least satisfy the valence state of the metals present in the catalyst;

ii) catalysts of the formula:

Ni_{g}AₕBᵢDⱼO_{f}

wherein g is a number from 0.1 to 0.9, in many cases from 0.3 to 0.9, in other cases from 0.5 to 0.85, in some instances 0.6 to 0.8; h is a number from 0.04 to 0.9; i is a number from 0 to 0.5; j is a number from 0 to 0.5; and f is a number to at least satisfy the valence state of the metals in the catalyst; A is chosen from Ti, Ta, V, Nb, Hf, W, Y, Zn, Zr, Si and Al or mixtures thereof; B is chosen from La, Ce, Pr, Nd, Sm, Sb, Sn, Bi, Pb, Tl, In, Te, Cr, Mn, Mo, Fe, Co, Cu, Ru, Rh, Pd, Pt, Ag, Cd, Os, Ir, Au, Hg, and mixtures thereof; D is chosen from Ca, K, Mg, Li, Na, Sr, Ba, Cs, and Rb and mixtures thereof; and O is oxygen;

iii) catalysts of the formula:

MoₐEₖGₗO_{f}

wherein E is chosen from Ba, Be, Ca, Cr, Mn, Nb, Ta, Ti, Te, V, W and mixtures thereof; chosen from Al, Bi, Ce, Co, Cu, Fe, K, Mg, V, Ni, P, Pb, Sb, Si, Sn, Ti, U, and mixtures thereof; a=1; k is 0 to 2; 1=0 to 2, with the proviso that the total value of 1 for Co, Ni, Fe and mixtures thereof is less than 0.5; and f is a number to at least satisfy the valence state of the metals in the catalyst;

iv) catalysts of the formula:

VₘMoₙNbₒTeₚMe_{q}O_{f}

wherein Me is chosen from Ta, Ti, W, Hf, Zr, Sb and mixtures thereof; m is from 0.1 to 3; n is from 0.5 to 1.5; o is from 0 to 3; p is from 0.001 to 5; q is from 0 to 2; and f is a number to at least satisfy the valence state of the metals in the catalyst;

v) catalysts of the formula:

MoₐVᵣXₛYₜZᵤMᵥO_{f}

wherein X is at least one of Nb and Ta; Y is at least one of Sb and Ni; Z is at least one of Te, Ga, Pd, W, Bi and Al; M is at least one of Be, Fe, Co, Cu, Cr, Ti, Ce, Zr, Mn, Pb, Mg, Sn, Pt, Si, La, K, Ag and In; a=1.0 (normalized); r=0.05 to 1.0; s=0.001 to 1.0; t=0.001 to 1.0; u=0.001 to 0.5; v=0.001 to 0.3; and f is a number to at least satisfy the valence state of the metals in the catalyst.

If the catalyst is made using a conventional hydrothermal process it may have the formula:

Mo_{1.0}V_{0.25-0.45}Te_{0.10-0.16}Nb_{0.15-0.19}O_{d}

wherein d is a number to at least satisfy the valence state of the metals in the catalyst.

An implementation of an ODH catalyst material is a mixed metal oxide having the formula Mo₁V_{0.1-1}Nb_{0.1-1}Te_{0.01-0.2}X_{0-0.2}O_{f} wherein X is selected from Pd, Sb Ba, Al, W, Ga, Bi, Sn, Cu, Ti, Fe, Co, Ni, Cr, Zr, Ca and oxides and mixtures thereof, and f is a number to satisfy the valence state of the metals present in the catalyst.

An implementation of an ODH catalyst material is a mixed metal oxide that includes Mo, V, O, and iron (Fe). The molar ratio of Mo to V can be from 1:0.25 to 1:0.50 or from 1:0.30 to 1:0.45, or from 1:0.30 to 1:0.35, or from 1:0.35 to 1:0.45. The molar ratio of Mo to Fe can be from 1:0.25 to 1:5.5, or from 1:3 to 1:5.5, or from 1:4.25 to 1:4.75, or from 1:4.45 to 1:4.55, or from 1:0.1 to 1:1, or from 1:0.25 to 1:0.75, or from 1:0.4 to about 1:0.6, or about 1:0.4, or about 1:0.6, or from 1:1.3 to 1:2.2, or from 1:1.6 to 1:2.0, or from 1:1.80 to 1:1.90. Further, oxygen is present at least in an amount to satisfy the valency of any present metal oxides. The catalyst can have at least a portion of the Fe in the catalyst material present as Fe(III). The catalyst can have at least a portion of the Fe in the catalyst material present as amorphous iron. The catalyst can have at least a portion of the Fe in the catalyst material present as an iron oxide, an iron oxide hydroxide, or a combination thereof. The iron oxide can include an iron oxide selected from hematite (α-Fe₂O₃), maghemite (γ-Fe₂O₃), magnetite (Fe₃O₄), or a combination thereof. The iron oxide hydroxide can include an iron oxide hydroxide selected from a goethite, an akageneite, a lepidocrocite, or a combination thereof. The catalyst can include at least a portion of the iron as a goethite and at least a portion of the iron as a hematite.

An implementation of an ODH catalyst material is a mixed metal oxide having the formula Mo₁V_{0.1-1}Nb_{0.1-1}Te_{0.01-0.2}X_{0-0.2}O_{f} wherein X is selected from Pd, Sb, Ba, Al, W, Ga, Bi, Sn, Cu, Ti, Fe, Co, Ni, Cr, Zr, Ca and oxides and mixtures thereof, and f is a number to satisfy the valence state of the metals present in the catalyst.

An implementation of an ODH catalyst material is a mixed metal oxide that includes Mo, V, O, and iron (Fe). The molar ratio of Mo to V can be from 1:0.25 to 1:0.50 or from 1:0.30 to 1:0.45, or from 1:0.30 to 1:0.35, or from 1:0.35 to 1:0.45. The molar ratio of Mo to Fe can be from 1:0.25 to 1:5.5, or from 1:3 to 1:5.5, or from 1:4.25 to 1:4.75, or from 1:4.45 to 1:4.55, or from 1:0.1 to 1:1, or from 1:0.25 to 1:0.75, or from 1:0.4 to about 1:0.6, or about 1:0.4, or about 1:0.6, or from 1:1.3 to 1:2.2, or from 1:1.6 to 1:2.0, or from 1:1.80 to 1:1.90. Further, oxygen is present at least in an amount to satisfy the valence state of the metals present in the catalyst. The catalyst can have at least a portion of the Fe in the catalyst material present as Fe(III). The catalyst can have at least a portion of the Fe in the catalyst material present as amorphous iron. The catalyst can have at least a portion of the Fe in the catalyst material present as an iron oxide, an iron oxide hydroxide, or a combination thereof. The iron oxide can include an iron oxide selected from hematite (α-Fe₂O₃), maghemite (γ-Fe₂O₃), magnetite (Fe₃O₄), or a combination thereof. The iron oxide hydroxide can include an iron oxide hydroxide selected from a goethite, an akageneite, a lepidocrocite, or a combination thereof. The catalyst can include at least a portion of the iron as a goethite and at least a portion of the iron as a hematite.

An implementation of an ODH catalyst material is a mixed metal oxide having the empirical formula Mo₁V_{0.25-0.5}O_{d} wherein d is a number to satisfy the valence state of the metals present in the catalyst. The molar ratio of Mo to V can be from 1:0.25 to 1:0.5, or 1:0.3 to 1:0.49.

An implementation of an ODH catalyst material is a mixed metal oxide that includes Mo, V, O, and aluminum (Al). The molar ratio of Mo to V can be from 1:0.1 to 1:0.50, or from 1:0.25 to 1:0.50, or from 1:0.3 to 1:0.49, or from 1:0.30 to 1:0.45, or from 1:0.30 to 1:0.35, or from 1:0.35 to about 1:0.45. The molar ratio of Mo to Al is from 1:1.5 to 1:6.5, or from 1:3.0 to 1:6.5, or from 1:3.25 to 1:5.5.5, or from 1:3.5 to 1:4.1, or from 1:4.95 to 1:5.05, or from 1:4.55 to 1:4.65, or from 1:1.5 to 1:3.5, or from 1:2.0 to 1:2.2, or from 1:2.9 to 1:3.1. Oxygen is present at least in an amount to satisfy the valence state of the metals present in the catalyst. At least a portion of the Al in the catalyst material can be present as an aluminum oxide; the aluminum oxide can be an aluminum oxide hydroxide. The aluminum oxide hydroxide can include an aluminum oxide hydroxide selected from a gibbsite, a bayerite, a boehmite, or a combination thereof. At least a portion of the Al in the catalyst material can be present as gamma alumina.

An implementation of an ODH catalyst material is a mixed metal oxide that includes Mo, V, O, Al, and Fe. The molar ratio of Mo to V can be from 1:0.1 to 1:0.5, or from 1:0.30 to 1:0.45, or from 1:0.30 to 1:0.35, or from 1:0.35 to 1:0.45. The molar ratio of Mo to Al can be from 1:1.5 to 1:6.0. The molar ratio of Mo to Fe can be from 1:0.25 to 5:5. Oxygen is present at least in an amount to satisfy the valence state of the metals present in the catalyst. The molar ratio of Mo to Fe can be from 1:0.1 to 1:1, and the molar ratio of Mo to Al can be from 1:3.5 to 1:5.5. The molar ratio of Mo to Fe can be from 1:0.25 to 1:0.75, and the molar ratio of Mo to Al can be from 1:3.75 to 1:5.25. The molar ratio of Mo to Fe can be from 1:0.35 to 1:0.65, and the molar ratio of Mo to Al can be from 1:3.75 to 1:5.25. The molar ratio of Mo to Fe can be from 1:0.35 to 1:0.45, and the molar ratio of Mo to Al can be from 1:3.9 to 1:4.0. The molar ratio of Mo to Fe can be from 1:0.55 to 0:65, and the molar ratio of Mo to Al can be from 1:4.95 to 1:5.05. The molar ratio of Mo to Fe can be from 1:1.3 to 1:2.2, and the molar ratio of Mo to Al can be from 1:2.0 to 1:4.0. The molar ratio of Mo to Fe can be from 1:1.6 to 1:2.0, and the molar ratio of Mo to Al can be from 1:2.5 to 1:3.5. The molar ratio of Mo to Fe can be from 1:1.80 to 1:1.90, and the molar ratio of Mo to Al can be from 1:2.9 to 1:3.1. At least a portion of the Fe in the catalyst material can be present as Fe(III). At least a portion of the Fe in the catalyst material can be present as amorphous Fe. At least a portion of the Fe in the catalyst material can be present as an iron oxide, an iron oxide hydroxide, or a combination thereof. In some embodiments, the iron oxide includes an iron oxide selected from hematite (α-Fe₂O₃), maghemite (γ-Fe₂O₃), magnetite (Fe₃O₄), or a combination thereof. Iron oxide hydroxide can include an iron oxide hydroxide selected from a goethite, an akageneite, a lepidocrocite, or a combination thereof. At least a portion of the Fe in the catalyst material can be present as a goethite and at least a portion of the Fe in the catalyst material can be present a hematite. At least a portion of the Al in the catalyst material can be is present as an aluminum oxide. The aluminum oxide can include an aluminum oxide hydroxide. The aluminum oxide hydroxide can include an aluminum oxide hydroxide selected from a gibbsite, a bayerite, a boehmite, or a combination thereof. At least a portion of the aluminum in the catalyst material can be present as a gamma alumina.

An implementation of an ODH catalyst material is a mixed metal oxide that includes Mo, V, Be, and O. The molar ratio of Mo to V can be from 1:0.25 to 1:0.65, or from 1:0.35 to 1:0.55, or from 1:0.38 to 1:0.48. The molar ratio of Mo to Be can be from 1:0.25 to 1:0.85, or from 1:0.35 to 1:0.75, or from 1:0.45 to 1:0.65. Oxygen is present at least in an amount to satisfy the valence state of the metals present in the catalyst.

An implementation of an ODH catalyst material is a mixed metal oxide that includes Mo, V, Be, Al and O. The molar ratio of Mo to V can be from 1:0.25 to 1:0.65, or from 1:0.35 to 1:0.55, or from 1:0.38 to 1:0.48. The molar ratio of Mo to Be can be from 1:0.25 to 1:1.7, or from 1:0.35 to 1:0.75, or from 1:0.45 to 1:0.65. The molar ratio of Mo to Al can be from 1:1 to 1:9, or from 1:2 to 1:8, or from 1:4 to 1:6. Oxygen is present at least in an amount to satisfy the valence state of the metals present in the catalyst. At least a portion of the aluminum in the catalyst material can be present as an aluminum oxide. The aluminum oxide can include an aluminum oxide hydroxide. The aluminum oxide hydroxide can include an aluminum oxide hydroxide selected from a gibbsite, a bayerite, a boehmite, or a combination thereof. At least a portion of the aluminum in the catalyst material can be present as gamma alumina.

An implementation of an ODH catalyst material has an amorphous phase of from about 20 wt. % to about 50 wt. %, or from about 25 wt. % to about 45 wt. %, or from about 45 wt. % to about 75 wt. %, or from about 55 wt. % to about 65 wt. %, or from about 50 wt. % to about 85 wt. %, or from about 55 wt. % to about 75 wt. %, or from about 60 wt. % to about 70 wt. %.

An implementation of an ODH catalyst material has an average crystallite size of greater than about 50 nm, or greater than about 75 nm, or greater than about 100 nm, or greater than about 125 nm, or from about 75 nm to about 150 nm, or from about 75 nm to about 250 nm, or from about 125 nm to about 175 nm.

An implementation of an ODH catalyst material has a mean particle size from about 0.5 μm to about 10 μm, or from about 2 μm to about 8 μm, or from about 3 μm to about 5 μm, or from about 0.5 μm to about 20 μm, or from about 5 μm to about 15 μm, or from about 7 μm to about 11 μm.

An implementation of an ODH catalyst material is characterized by having at least one or more XRD diffraction peaks (20 degrees) chosen from 6.5±0.2, 7.8±0.2, 8.9±0.2, 10.8±0.2, 13.2±0.2, 14.0±0.2, 22.1±0.2, 23.8±0.2, 25.2±0.2, 26.3±0.2, 26.6±0.2, 27.2±0.2, 27.6±0.2, 28.2±0.2, 29.2±0.2, 30.5±0.2, and 31.4±0.2 wherein the XRD is obtained using CuKα radiation. An implementation of an ODH catalyst material is characterized by having at least one or more XRD diffraction peaks (20 degrees) chosen from 6.6±0.2, 6.8±0.2, 8.9±0.2, 10.8±0.2, 13.0±0.2, 22.1±0.2, 26.7±0.2, 27.2±0.2, and 28.2±0.2, wherein the XRD is obtained using CuKα radiation.

An implementation of an ODH catalyst material can include from about 0.8 wt. % to about 30 wt. % calcium. The catalyst material can include about 0.15 wt. % to about 2.8 wt. % calcium. The catalyst material can include about 0.5 wt. % to about 75 wt. % calcium carbonate. The catalyst material can include about 5 wt. % to about 15 wt. % calcium carbonate.

The catalyst may be supported on or agglomerated with a binder, carrier, diluent or promoter. Some binders include acidic, basic or neutral binder slurries of TiO₂, ZrO₂, Al₂O₃, AlO(OH) and mixtures thereof. Another useful binder includes Nb₂O₅. The agglomerated catalyst may be extruded in a suitable shape (rings, spheres, saddles, etc.) of a size typically used in fixed bed reactors. When the catalyst is extruded, various extrusion aids known in the art can be used. In some cases, the resulting support may have a cumulative surface area of as high as 300 m²/g as measured by BET, in some cases less than about 35 m²/g, in some cases, less than about 20 m²/g, in other cases, less than about 3 m²/g, and a cumulative pore volume from about 0.05 to about 0.50 cm³/g.

The catalysts may be alone or in combination. Also, in some embodiments the catalysts may be used with a promoter such ad Pd, Pt or Ru to increase the catalyst activity. In relative terms the catalyst used in the first up to 40% of the initial length of the catalyst bed in the direction of flow of the reactants with having a reactivity not more than about 90% in some instances not more than about 80% of the reactivity of the average catalyst capacity in the remaining length of the catalyst bed.

The mixed metal oxide catalyst can be a supported catalyst. The support may be selected from oxides of titanium, zirconium, aluminum, magnesium, yttrium, lanthanum, silicon, zeolites and clays and their mixed compositions or a carbon matrix. The mixed metal oxide catalyst can also have a binder added which increases cohesion among the catalyst particles and optionally improves adhesion of the catalyst to the support if present. The mixed metal oxide catalyst can be diluted with inert material, such as DENSTONE® 99 alumina particles or SS 316 particles.

The mixed metal oxide catalyst either with or without a support can have a length to diameter ratio of 1:1 up to 10:1, in some cases with a length to diameter ratio of 1:1 to 5:1. The mixed metal oxide catalyst either with or without a support can be spherical, cylindrical, slab shaped, or any other shape. The mixed metal oxide catalyst either with or without a support can include particles that have notches on each end of each cylinder, in some embodiments up to 3 notches on the end of each cylinder. The mixed metal oxide catalyst either with or without a support can also contain one or several external “bumps” or protuberances which can be continuous and extend the length of the particle. The mixed metal oxide catalyst can be shaped in the form of hollow cylinders or rings. The mixed metal oxide catalyst either with or without a support can contain at least one passage through each particle. A person skilled in the art would know which features are required with respect to shape and dimensions of the mixed metal oxide catalyst.

### EXAMPLES

A fixed bed reactor unit (FBRU) apparatus was used to conduct experiments on the oxidative dehydrogenation of ethane. The FBRU apparatus comprised two vertically oriented fixed bed tubular reactors in series, each reactor a SS316L tube with an outer diameter of 1″ and a length of 34″, wrapped in an electrical heating jacket and sealed with ceramic insulating material. Each reactor contained an identical catalyst bed consisting of 143 g of a catalyst of the formula, as measured by PIXE analysis, of MoV_{0.30-0.40}Te_{0.10-0.20}Nb_{0.10-0.20}O_{X}, in which X was calculated based on the highest oxidation state of the metal oxides present in this catalyst, with relative atomic amounts of each component, relative to a relative amount of Mo of 1, shown in subscript. The 35% conversion temperature of the catalyst was ˜380° C. as measured in an MRU setup using 2 g of catalyst and a feed composition of 36/18/46 vol. % of ethane, oxygen, and nitrogen, respectively, at a feed gas flow rate of 154 sccm and atmospheric outlet pressure.

Both reactors, above and below the catalyst bed were packed with quartz powder secured in place with glass wool to minimize risk of catalyst bed movement during the experimental runs.

Experiments included runs using a feed stream comprising the components ethane, carbon dioxide, and water, pre-mixed and heated to a temperature of less than or equal to about 220° C. before introduction into the first reactor. The output from the first reactor was transferred to the second reactor without adding additional components and the same temperature was maintained for each reactor. The temperature of each of the catalyst beds in each reactor was monitored using four thermocouples located at points equally spaced along the length of each bed. The highest temperature between thermocouple points in each bed was used for controlling the reactor temperature using a corresponding back pressure regulator that controlled the pressure and boiling temperature of water inside reactor water jackets surrounding each reactor. The reaction temperature for each reactor was calculated as an average of all 8 points.

A simulation of an ODH reactor was developed using gPROMS ProcessBuilder® 1.2.0. The SRK equation of state was used to define component properties in Multiflash. The kinetic model for the ODH reaction was developed in gPROMS ProcessBuilder 1.2.0 and the kinetic parameters were estimated using fixed bed reactor data, from the FBRU experiments described above. The mixed metal oxide catalyst used was MoₐV_{b}Te_{c}Nb_{d}Oₑ, wherein a, b, c, d, and e are the relative atomic amounts of the elements Mo, V, Te, Nb, and O, respectively; and when a=1, b=0.01 to 1.0, c=0.01 to 1.0, d=0.01 to 1.0, and e is a number to satisfy the valence state of the catalyst. Table 1 shows the comparison of FBRU experimental data at 360° C. with the model predictions. The model predictions are in good agreement with the reactor data.

TABLE 1

| | Reactor Data Mass | Model Prediction Mass |
|---|---|---|
| Components | Fraction | Fraction |
| H₂O | 0.296 | 0.305 |
| C₂H₆ | 0.141 | 0.140 |
| C₂H₄ | 0.114 | 0.114 |
| O₂ | 0.025 | 0.023 |
| CO₂ | 0.388 | 0.387 |
| CO | 0.020 | 0.016 |
| AA | 0.015 | 0.015 |
| Conversion (%) | 51.0 | 51.1 |
| Selectivity (wt. %) | 77.7 | 77.9 |
| Yield (wt. %) | 39.6 | 39.8 |

The following examples demonstrate the effect of changing the catalyst capacity profile, either by changing the dilution ratio or the void fraction, on the maximum process temperature. For each simulation example the mass flow rate of the feed of each of the components to the simulated ODH reactor was consistent and is shown in Table 2. The simulated feed temperature and pressure were also consistent, at 350° C. and 196.5 kPa, respectively. Table 3 shows the simulated thermophysical properties of the ODH catalyst. Results for each simulation example are shown in Table 4. Reactor dimensions were altered to maintain the same amount, in g, of catalyst throughout the catalyst bed. The total amount of catalyst in each example was set to 197.9 g.

TABLE 2

| Components | Unit | Value |
|---|---|---|
| C₂H₆ | kg/hr | 0.082 |
| O₂ | kg/hr | 0.044 |
| CO₂ | kg/hr | 0.105 |
| H₂O | kg/hr | 0.057 |
| CH₃COOH | kg/hr | 0.00 |
| Total | kg/hr | 0.288 |

TABLE 3

| Property | Unit | Value |
|---|---|---|
| Bulk Density | kg/m³ | 817 |
| Heat Capacity | J/(kg · K) | 880 |
| Pellet Conductivity | W/mK | 14.3 |

### Example 1 (Comparative 1)

Example 1 simulation conditions included 197.9 g of active catalyst and a dilution ratio of 0.55, the catalyst particles having a cylindrical shape with an average length and diameter of 5 mm and 3.175 mm, respectively. The void fraction was set to 0.421. The length of the simulated reactor was 2.7 m, with an outside diameter of 25.4 mm and a wall thickness of 2.1 mm. The coolant inlet temperature was set to be similar to that of the feed, i.e. 350° C., and the outlet temperature was 352° C. The wall-coolant heat transfer coefficient was set to be 1000 W/m²K. The results are shown in Table 4.

### Example 2 (Comparative 2)

Example 2 followed the same simulated conditions, void fraction, and ODH catalyst shape and size as Example 1. The simulation also included 197.9 g of active catalyst, but only occupying 40 vol. % of the catalyst bed. To reduce the active catalyst vol. % while maintaining the same amount of catalyst active phase the length of the reactor was increased to 3.0 m (in effect increasing the dilution ratio) while keeping outside diameter at 25.4 mm and wall thickness at 2.1 mm. The coolant inlet temperature was set to be similar to that of the feed, i.e. 350° C. and the outlet temperature was 352° C. For this case, the wall-coolant heat transfer coefficient was set to be 470 W/m²K. The results are shown in Table 4.

The comparative results demonstrate that the maximum process temperature, and the temperature gradient, may be decreased by loading a similar amount of catalyst in a larger reactor. Both examples include the same amount of catalyst but in Example 2 the catalyst is distributed within a larger volume. Increasing the volume of the reactor may increase the cost of construction.

### Example 3

Example 3 also used the same simulated conditions, void fraction, and ODH catalyst shape and size as Example 1. The simulation included fractioning the catalyst bed into two sections. The upstream section covering the first 70% of catalyst bed length and having 133.5 g of catalyst and a dilution ratio of 0.40, and the downstream section covering the final 30% of the catalyst bed length and having 64.4 g of catalyst and a 0.55 dilution ratio. To maintain the same amount of total catalyst in the reactor as in Example 1, the length of the reactor was increased to 2.9 m from of 2.7 m. The outside diameter and wall thickness remained the same. The coolant inlet temperature was assumed to be similar to that of the feed, i.e. 350° C. and the outlet temperature is 352° C. For this case, the wall-coolant heat transfer coefficient was set to be 1000 W/m²K. The results shown in Table 4 show a decrease in the maximum process temperature, seen in the upstream section, of about 10.8° C. and 2.6° C., as compared to both example 1 and 2, respectively.

### Example 4

In Example 4 the dilution ratio was set to 0.55 across the length of the catalyst bed. Similar to example 3 the catalyst bed was divided into two sections, with the upstream section covering the first 70% of the catalyst bed length and the downstream section covering the remaining 30% of the catalyst bed length. The void fraction for the upstream section was increased to 0.436, compared to 0.421 for the downstream section, by adjusting the catalyst shape to particles having a length of 7.5 mm and a diameter of 4.8 mm. The downstream section was set with catalyst particles of length 5.0 mm and diameter 3.2 mm. The upstream section was to set include 137.5 g of catalyst and the downstream section was set to include 60.4 g of catalyst, for a total of 197.9 g. The length of the catalyst bed was set to 2.7 m, the outside diameter was set to 25.4 mm, and the wall thickness was set to 2.1 mm. The coolant inlet temperature was assumed to be similar to that of the feed, i.e. 350° C. and the outlet temperature is 352° C. For this case, the wall-coolant heat transfer coefficient was set to be 1000 W/m²K. The results are shown in Table 4.

TABLE 4

| | Ex. 1 | Ex. 2 | Ex. 3 | | Ex. 4 | |
|---|---|---|---|---|---|---|
| | Section | | | | | |
| | | | Up | Down | Up | Down |
| | n/a | n/a | Stream | Stream | Stream | Stream |
| Catalyst Bed Length (m) | 2.74 | 3.05 | 2.93 | | 2.93 | |
| Fraction of the Catalyst | 1.0 | 1.0 | 0.7 | 0.3 | 0.7 | 0.3 |
| Bed (reactor) Length | | | | | | |
| Dilution Ratio | 0.55 | 0.60 | 0.60 | 0.55 | 0.55 | 0.55 |
| Mass of Catalyst (g) | 197.9 | 197.9 | 133.5 | 64.4 | 137.5 | 60.4 |
| Catalyst Particle | 5.0 | 5.0 | 5.0 | 5.0 | 7.5 | 5.0 |
| Length (mm) | | | | | | |
| Catalyst Particle | 3.2 | 3.2 | 3.2 | 3.2 | 4.8 | 3.2 |
| Diameter (mm) | | | | | | |
| Void fraction | 0.421 | 0.421 | 0.421 | 0.421 | 0.436 | 0.421 |
| Coolant Outlet Temperature | 351.2 | 352.2 | 350.6 | 352.0 | 351.0 | 352.0 |
| Max Process Temp Tₘ | 379.2 | 371.0 | 368.4 | 352.9 | 366.3 | 353.5 |
| Differential Temp (° C.) | 28.0 | 18.8 | 17.8 | 0.9 | 15.3 | 1.5 |
| Average Temp Tₐᵥ (° C.) | 356.7 | 356.1 | 355.5 | 353.1 | 356.0 | 353.4 |
| Temp Isotherm T_{c} (° C.) | 352.5 | 353.4 | 352.3 | 353.4 | 352.9 | 353.4 |
| Inlet Pressure (kPa) | 196.47 | 196.47 | 196.47 | 169.09 | 196.47 | 169.70 |
| Outlet Pressure (kPa) | 136.15 | 135.81 | 169.09 | 135.87 | 169.70 | 136.69 |
| Pressure Drop (kPa) | 60.33 | 60.66 | 27.39 | 33.22 | 26.78 | 33.01 |

| | | | | |
|---|---|---|---|---|
| Conversion (%) | 54.9 | 55.1 | 55.8 | 55.2 |
| Selectivity (mol. %) | 79.0 | 79.2 | 80.0 | 79.4 |

The effect of differing catalyst capacity profiles from the preceding examples on the temperature profiles in the simulated ODH reactor are presented FIG. 10. Particularly, FIG. 10 illustrates Temperature Profiles of the examples described. All four Example lines show a maximum value of process temperature (y-axis) that occurs within the first 10% of the catalyst bed, shown as Dim. Reactor Length (x-axis).

It can be seen that by manipulating either the dilution ratio or the void fraction, in effect changing the volume fraction of active phase in the catalyst and changing the catalyst dimensions, the temperature profile is changed. Even with small changes in the dilution ratio (8.3% decrease from the upstream to the downstream section) or void fraction (3.5% decrease from the upstream to the downstream section) a change in the maximum process temperature was observed, reduced by 10.8° C. and 12.9° C., respectively. Also, the temperature differential decreased by 10.2° C. and 13.7° C. in relation to changes in the dilution ratio and void fraction, respectively. Larger changes in catalyst capacity between the sections would likely reduce the maximum process temperature and temperature differential further.

Considering the axial temperature profile in the tubular reactor, the maximum process temperature occurs close to the inlet of the reactor, which is between 0-20% of the length of the reactor. The temperature profile gradually drops off to an isotherm towards the end of the tubular reactor. About 70% of the ethane conversion achieved inside the tubular reactor occurs within 20% of the inlet of the simulated reactor. This maximum process temperature also impacts ethylene selectivity. Ethylene selectivity may drop as the difference between the maximum process temperature and the temperature isotherm increases. While the selectivity remains relatively unchanged in the above examples, it is expected that with larger changes in the catalyst capacity the difference in the max temperature and the isotherm will reduce further, likely improving selectivity. In addition, if the maximum process temperature is not properly controlled, it can result in hot spot inside the reactor. In order to control this maximum process temperature, the coolant flow on the shell side of the reactor can be manipulated.

These examples exemplify a method to control or reduce the maximum process temperature within a first section (up to 50 vol. %) of an oxidative dehydrogenation reactor catalyst bed, the oxidative dehydrogenation reactor converting some of a feed stream of ethane to ethylene, and to shift the location of the maximum process temperature in a direction contrary to the flow of feed of reactants and heat removal diluent gas. This may be done by using a catalyst bed having a lower catalyst capacity in in the first section of the reactor (reactivity per volume lower than that of the remaining section or sections of the catalyst bed).

The detailed description, embodiments, and examples provided herein are intended for illustrative purposes only and not intended to limit the scope of the present disclosure, which should be understood to include various additional aspects, modifications or changes that would be apparent to those skilled in the art.

### INDUSTRIAL APPLICABILITY

The present disclosure relates to a fixed bed reactor system for use in an ethane oxidative dehydrogenation process.

1. A fixed bed reactor system for the oxidative dehydrogenation (ODH) of ethane to ethylene comprising a catalyst bed, wherein a catalyst capacity profile increases, gradually or in steps, from an upstream end to a downstream end of the catalyst bed.

2. The fixed bed reactor system of claim 1 wherein the catalyst bed comprises at least two non-overlapping catalyst bed sections arranged in series along the catalyst bed length, wherein the catalyst bed sections are identified by a change in the catalyst capacity, and wherein each catalyst bed section has a higher catalyst capacity than the preceding upstream catalyst bed section.

3. The fixed bed reactor system of claim 2, wherein one or more catalyst bed sections comprise a lower 35% conversion temperature than the preceding catalyst bed section.

4. (canceled)

5. The fixed bed reactor system of claim 4, wherein one or more catalyst bed sections comprise a 35% conversion temperature that is from 2 to 10° C. lower than the preceding catalyst bed section.

6. The fixed bed reactor system of claim 2, wherein one or more catalyst bed sections comprise a dilution ratio that is lower than the preceding catalyst bed section.

7-9. (canceled)

10. The fixed bed reactor system of claim 6, wherein one or more catalyst bed sections comprise a dilution ratio that is from 2 to 15% lower than the preceding catalyst bed section.

11-13. (canceled)

14. The fixed bed reactor system of claim 11, wherein one or more catalyst bed sections comprise a void fraction that is from 10 to 25% lower than the preceding catalyst bed section.

15. A process for the oxidative dehydrogenation (ODH) of ethane to ethylene comprising:
introducing a feed stream comprising ethane and oxygen into a fixed bed reactor system comprising:
a catalyst bed, wherein an ODH catalyst capacity profile increases along the length of the catalyst bed from the upstream end to the downstream end;
contacting the ethane and the ODH catalyst in the presence of oxygen along the catalyst bed length to convert at least a fraction of the ethane into ethylene; and
removing a product stream comprising ethylene from the ODH reactor in close proximity to the downstream end of the catalyst bed.

16. A method to reduce a maximum reaction temperature within a first section of an oxidative dehydrogenation reactor catalyst bed comprised of at least one mixed metal oxide catalyst, the oxidative dehydrogenation reactor comprising at least one feed stream and at least one outlet stream, the catalyst bed comprised of at least two catalyst bed sections, a first catalyst bed section upstream of subsequent catalyst bed sections, wherein the first catalyst bed section is less than or equal to 50 vol % of the catalyst bed, the method comprising one or more of:
i). a higher dilution ratio in the first catalyst bed section than in subsequent catalyst bed sections, such that the dilution ratio in each of the subsequent catalyst bed sections divided by the dilution ratio in the first catalyst bed section is in the range of 0.3 to 0.98; and
ii). a higher void fraction in the first catalyst bed section than in subsequent catalyst bed sections, such that the ratio of void fraction in each of the subsequent catalyst bed sections divided by the void fraction in the first catalyst bed section is in the range of 0.3 to 0.98.

17. The method of claim 16, wherein the catalyst bed comprises heat dissipative particles.

18. The method of claim 17, wherein the heat dissipative particles are comprised of inert metal rods.

19. The method of claim 17, wherein the heat dissipative particles are comprised of DENSTONE® 99 particles.

20. The method of claim 17, wherein the heat dissipative particles are comprised of SS 316.

21-22. (canceled)

23. The method of claim 17, wherein the heat dissipative particles are comprised of particles with a particle size in the range of 0.5 to 5 mm.

24-25. (canceled)

26. The method of claim 16, wherein the catalyst bed is comprised of particles with particle size in the range of 0.5 to 5 mm.

27. The method of claim 16, wherein the mixed metal oxide catalyst is comprised of particles with particle size in the range of 0.5 μm to 20 μm.

28. (canceled)

29. The method of claim 16, wherein the dilution ratio in each of the subsequent catalyst bed sections divided by the dilution ratio in the first catalyst bed section is in the range of 0.85 to 0.98.

30. The method of claim 16, wherein the ratio of void fraction in each of the subsequent catalyst bed sections divided by the void fraction in the first catalyst bed section is in the range of 0.35 to 0.55.

31. (canceled)

32. The method of claim 16, wherein the mixed metal oxide catalyst is comprised of particles with a length to diameter ratio of 1:1 to 5:1.

33. The method of claim 16, wherein the mixed metal oxide catalyst is comprised of particles that contain at least one passage through each particle.

34. The method of claim 16, wherein the mixed metal oxide catalyst is comprised of particles that are cylindrical.

35-36. (canceled)

37. The method of claim 16, wherein the oxidative dehydrogenation reactor comprises at least two sections of catalyst bed comprised of at least one mixed metal oxide catalyst, the at least one feed stream to the oxidative dehydrogenation reactor comprising oxygen and not less than 20 vol. % of ethane, the at least one outlet stream comprising one or more ethylene, ethane, one or more carboxylic acids, water and oxygen.

38-40. (canceled)

41. The method of claim 16, wherein the temperature difference between the maximum reaction temperature within a first section of an oxidative dehydrogenation reactor catalyst bed and the temperature in subsequent catalyst bed sections is from 5 to 20° C.

42-44. (canceled)

45. The method of claim 16, wherein the maximum reaction temperature is from 310° C. to 350° C.

46. (canceled)

47. The method of claim 16, wherein the oxidative dehydrogenation reactor is at a pressure from 15 to 50 psig.

48. (canceled)

49. The method of claim 16, wherein the oxidative dehydrogenation reactor has a residence time from 0.1 to 10 seconds.

50. (canceled)

51. The method of claim 16, wherein the oxidative dehydrogenation reactor has a gas hourly space velocity from 500 to 3,000 h⁻¹.

52. The method of claim 16, wherein at least one oxidative dehydrogenation reactor comprises a fixed bed type reactor.

53-56. (canceled)

57. A method for loading a catalyst bed in a fixed bed reactor for oxidative dehydrogenation of ethane, the fixed bed reactor comprising an upstream end and a downstream end, the method comprising;
preparing two or more catalyst bed compositions, the catalyst bed compositions comprising an ODH catalyst;
determining a catalyst capacity for each of the catalyst bed compositions;
separately pouring, in sequential order, the catalyst bed compositions into the fixed bed reactor at a rate slow enough to allow dense and random packing, with the catalyst bed composition having the lowest catalyst capacity poured into the upstream end and the catalyst bed composition having the highest catalyst capacity poured into the downstream end; and
securing the poured catalyst bed compositions within the fixed bed reactor to form a loaded catalyst bed; and
wherein the catalyst bed compositions form distinct catalyst bed sections, the catalyst bed sections identified by the change in catalyst capacity and increasing from the upstream end to the downstream end.

58. (canceled)

59. The method of claim 57 wherein catalyst capacity is assessed by determining a 35% conversion temperature for each of the catalyst bed compositions, and ordering catalyst bed compositions with the highest relative 35% conversion temperature corresponding to the catalyst bed composition with the lowest relative catalyst capacity.
